(19) United States
(12) Patent Application Publication
(10) Pub. No.: US 2023/0121183 A1
(43) Pub. Date: Apr. 20, 2023

(54) SENSOR BANDAGE AND METHOD FOR MAKING A SENSOR BANDAGE WITH TUNED FLEXIBILITY

(71) Applicant: Covidien LP, Mansfield, MA (US)

(72) Inventor: Sarah L. Hayman, Boulder, CO (US)

(21) Appl. No.: 17/966,570

(22) Filed: Oct. 14, 2022

Related U.S. Application Data
(60) Provisional application No. 63/255,650, filed on Oct. 14, 2021.

(51) Int. Cl. A61B 5/00

(52) U.S. Cl. CPC A61B 5/6833

(57) ABSTRACT

A bandage for a medical sensor or system is described, the bandage including plural transparent wings surrounding at least one medical sensor, the plural transparent wings including an adhesive on a first side and including apertures therethrough configured to provide stretchability in at least one direction to the sides and away from the at least one medical sensor.

### CROSS-REFERENCE TO RELATED APPLICATIONS

This application claims the benefit of U.S. Provisional Application No. 63/255,650, filed Oct. 14, 2021, the disclosure of which is incorporated herein by reference in its entirety.

### FIELD

The present disclosure relates generally to medical devices, and more particularly, to medical devices, such as pulse oximeters, that monitor physiological parameters of a patient applied to the patient using a sensor bandage.

### BACKGROUND

In the field of medicine, doctors often desire to monitor certain physiological characteristics of their patients. Accordingly, a wide variety of devices have been developed for monitoring many such physiological characteristics. Such devices provide doctors and other healthcare personnel with the information they need to provide the best possible healthcare for their patients. As a result, such monitoring devices have become an indispensable part of modern medicine.

One technique for monitoring certain physiological characteristics of a patient uses attenuation of light to determine physiological characteristics of a patient. This is used in pulse oximetry, and the devices built based upon pulse oximetry techniques. Light attenuation is also used for regional or cerebral oximetry. Oximetry may be used to measure various blood characteristics, such as the oxygen saturation of hemoglobin in blood or tissue, the volume of individual blood pulsations supplying the tissue, and/or the rate of blood pulsations corresponding to each heartbeat of a patient. The signals can lead to further physiological measurements, such as respiration rate, glucose levels or blood pressure.

One issue in such sensors relates to patient swelling at the sensor site, for example when applied to a patient digit, such as a finger or toe. This can cause retention of fluid at the site of the bandage and patient discomfort. In the case of a bandage applied to a finger, for example, fluid retention can cause the finger to swell, with concurrent tightening of the bandage on the finger.

Additionally, a patient can become diaphoretic and/or the bandage can become moist. With general regard to wound care, e.g., U.S. Pat. No. 7,612,248, assigned to 3M, describes various configurations for perforated patient facing layers in conjunction with a backing layer with an absorbent substrate to draw exudate into the absorbent layer. However, bandages for medical sensors are not constructed with a view to drawing exudate from a wound into an absorbent layer.

For traditional medical sensor bandages, if the bandage cannot adequately allow moisture to evaporate, the sensor's patient side adhesive can become stressed when the finger swells and becomes wet, such that the adhesive shears and does not stay adhered to the patient's finger. In such circumstances, the sensor can become like a “boot”, such that the patient adhesive is not stuck to the patient, but the flaps of the bandage are still adhered to one another. When the sensor becomes like a “boot”, it can slide off the finger, causing monitoring errors.

What is needed in the art is a sensor bandage that alleviates these concerns, including issues surrounding swelling of the patient at the sensor bandage and prevention of failure of the patient adhesive, as described above.

### SUMMARY

The techniques of this disclosure generally relate to bandages for medical devices that monitor physiological parameters of a patient, such as pulse oximeters.

In one aspect, the present disclosure provides a bandage for a medical sensor, the bandage including plural transparent wings surrounding at least one medical sensor, the plural transparent wings including an adhesive on a first side and including apertures therethrough configured to provide stretchability in at least one direction to the sides and away from the at least one medical sensor.

In exemplary aspects, the apertures include longitudinal slits oriented in a direction perpendicular to the at least one direction of stretchability. In exemplary aspects, the slits are knife cut (with or without removal of material), laser cut (with removal of material), or other wised formed through the bandage material.

In further exemplary aspects, plural rows of longitudinal slits, e.g., 2-4 rows of slits are provided on each wing. In further exemplary aspects, the slits in the plural rows are staggered between rows. In further exemplary aspects, the slits are stacked (lined up) between rows. In exemplary aspects, the slits are provided at nominal lengths between 3 and 5 millimeters (mm), either all with the same length, or with different lengths on the same wings. Exemplary embodiments include, without limitations: slit lengths of 2 mm, 3 mm frequency, staggered; slit lengths of 2 mm, 4 mm frequency, staggered; slit lengths of 3 mm, 3 mm frequency, staggered; slit lengths of 5 mm, 2 mm frequency, staggered; and slit lengths of 5 mm, 3 mm frequency, staggered, though other slit lengths, frequencies and configurations (staggered vs. stacked), or mixtures thereof are contemplated herein.

In exemplary aspects, the slits are provided such that they do not cut through the edge of the bandage wings, thereby providing a constant outer bandage boundary that resists tearing during adhesive liner removal, application to a patient and adjustment/readjustment. In further exemplary embodiments, slit portions are spaced at a minimum distance away from the wing outer boundaries (those boundaries) e.g., more than about 1 mm, more than about 2 mm, more than about 2.5 mm, more than about 3 mm, more than about 3.5 mm, more than about 4 mm, more than about 4.5 mm, more than about 5 mm, etc.

In exemplary aspects, the sensor bandage includes wings providing stretchability up to 8 mm. In further exemplary aspects, the sensor bandage includes four wings, with each wing providing stretchability up to 2 mm to accommodate a total radius stretch of 8 mm. In further exemplary aspects, the material strain for the wings is up to about 0.2.

In exemplary aspects, the transparent material is a polyethylene material. In exemplary aspects, the thickness of the polyethylene material is between 0.07 and 0.12 mm thick). In further exemplary aspects, the material is a polyethylene material that is 0.09 mm thick. In further exemplary embodiments, the polyethylene material is acrylic coated. In further exemplary aspects, material is a 0.09 mm acrylic coated polyethylene material. In further exemplary aspects, the geometry of each wing is configured to provide the stretchability described above with an estimation of a 0.6 to 0.75 N axial force applied by a patient finger or other digit.

In further exemplary aspects, the geometry of each wing is configured to provide the deflection described above with an estimation of a 0.68 N axial force within the bandage applied by a patient finger (described further below). In further exemplary aspects, the 0.68 N axial force is based upon a diastolic blood pressure of 40 millimeters of mercury (mmHg) applying a hoop force on the bandage, with the bandage able to strain at least 0.2 at the force of 0.68 N. In other exemplary aspects, modeled deflection trends illustrated advantages for slit lengths of 5 mm, staggered, with either two or three rows, though other configurations are contemplated herein.

In further exemplary aspects, the slits provided in the plural wings additionally improve breathability of the bandage, mitigating issues with regard to moist bandages due to diaphoretic patients or other causes of moisture (spills, sweating in general, etc.).

In another aspect, the disclosure provides a patient monitoring system, having a patient monitor coupled to a patient monitoring sensor, which includes the sensor bandage described in exemplary aspects above. The patient monitoring sensor includes a communication interface, through which the patient monitoring sensor can communicate with the patient monitor. The patient monitoring sensor also includes a light-emitting diode (LED) communicatively coupled to the communication interface and a detector, communicatively coupled to the communication interface, capable of detecting light.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 illustrates a perspective view of an exemplary patient monitoring system including a patient monitor and a patient monitoring sensor;

FIG. 2 illustrates a top perspective view of a patient monitoring sensor, including a bandage in accordance with exemplary embodiments described herein;

FIG. 3 illustrates a side perspective view of the patient monitoring sensor of FIG. 2 secured in place over the finger of a patient;

FIG. 4A illustrates an exemplary schematic view of one exemplary slit orientation and pattern for a bandage wing in accordance with exemplary embodiments described herein;

FIG. 4B illustrates an exemplary schematic view of another exemplary slit orientation and pattern for a bandage wing in accordance with exemplary embodiments described herein;

FIG. 4C illustrates an exemplary schematic view of another exemplary slit orientation and pattern for a bandage wing in accordance with exemplary embodiments described herein;

FIG. 4D illustrates an exemplary schematic view of another exemplary slit orientation and pattern for a bandage wing in accordance with exemplary embodiments described herein;

FIG. 4E illustrates an exemplary schematic view of another exemplary slit orientation and pattern for a bandage wing in accordance with exemplary embodiments described herein; and

FIG. 5 illustrates an exemplary cross sectional view of forces applied by a swollen finger on a patient bandage.

### DETAILED DESCRIPTION

As has been noted, traditional pulse oximeters include bandage components that can cause discomfort related to patient swelling and moisture.

Accordingly, the present disclosure describes a medical sensor bandage that includes plural transparent wings surrounding at least one medical sensor, the plural transparent wings including an adhesive on a first side and including apertures therethrough configured to provide stretchability in at least one direction to the sides and away from the at least one medical sensor.

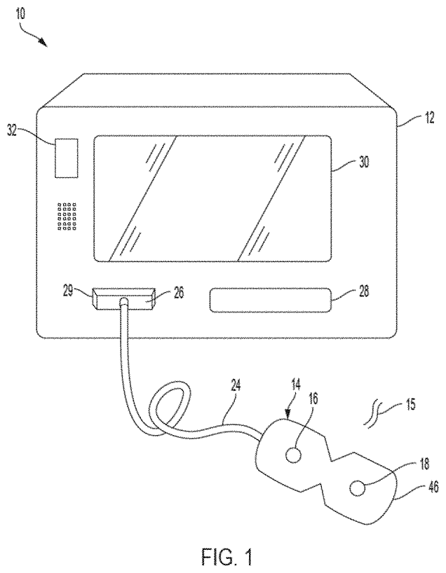

Referring now to FIG. 1, an embodiment of a patient monitoring system 10 that includes a patient monitor 12 and a sensor 14, such as a pulse oximetry sensor, to monitor physiological parameters of a patient is shown. By way of example, the sensor 14 may be a NELLCOR™, or INVOS™ sensor available from Medtronic (Boulder, Colo.), or another type of oximetry sensor. Although the depicted embodiments relate to sensors for use on a patient's fingertip, toe, or earlobe, it should be understood that, in certain embodiments, the features of the sensor 14 as provided herein may be incorporated into sensors for use on other tissue locations, such as the forehead and/or temple, the heel, stomach, chest, back, or any other appropriate measurement site.

In the embodiment of FIG. 1, the sensor 14 is a pulse oximetry sensor that includes one or more emitters 16 and one or more detectors 18. For pulse oximetry applications, the emitter 16 transmits at least two wavelengths of light (e.g., red and/or infrared (IR)) into a tissue of the patient. For other applications, the emitter 16 may transmit 3, 4, or 5 or more wavelengths of light into the tissue of a patient. The detector 18 is a photodetector selected to receive light in the range of wavelengths emitted from the emitter 16, after the light has passed through the tissue. Additionally, the emitter 16 and the detector 18 may operate in various modes (e.g., reflectance or transmission). In certain embodiments, the sensor 14 includes sensing components in addition to, or instead of, the emitter 16 and the detector 18. For example, in one embodiment, the sensor 14 may include one or more actively powered electrodes (e.g., four electrodes) to obtain an electroencephalography signal.

The sensor 14 also includes a sensor body 46 to house or carry the components of the sensor 14. The body 46 includes a backing, or liner, provided around the emitter 16 and the detector 18. Additionally, as will be further described, a bandage portion (not shown in FIG. 1, may be provided over or otherwise around the body with an adhesive to secure the sensor to the patient and to other portions of the sensor body 46.

In the embodiment shown, the sensor 14 is communicatively coupled to the patient monitor 12. In certain embodiments, the sensor 14 may include a wireless module configured to establish a wireless communication 15 with the patient monitor 12 using any suitable wireless standard. For example, the sensor 14 may include a transceiver that enables wireless signals to be transmitted to and received from an external device (e.g., the patient monitor 12, a charging device, etc.). The transceiver may establish wireless communication 15 with a transceiver of the patient monitor 12 using any suitable protocol. For example, the transceiver may be configured to transmit signals using one or more of the ZigBee standard, 802.15.4x standards WirelessHART standard, Bluetooth standard, IEEE 802.11x standards, or MiWi standard. Additionally, the transceiver may transmit a raw digitized detector signal, a processed digitized detector signal, and/or a calculated physiological parameter, as well as any data that may be stored in the sensor, such as data relating to wavelengths of the emitters 16, or data relating to input specification for the emitters 16, as discussed below. Additionally, or alternatively, the emitters 16 and detectors 18 of the sensor 14 may be coupled to the patient monitor 12 via a cable 24 through a plug 26 (e.g., a connector having one or more conductors) coupled to a sensor port 29 of the monitor. In certain embodiments, the sensor 14 is configured to operate in both a wireless mode and a wired mode. Accordingly, in certain embodiments, the cable 24 is removably attached to the sensor 14 such that the sensor 14 can be detached from the cable to increase the patient's range of motion while wearing the sensor 14.

The patient monitor 12 is configured to calculate physiological parameters of the patient relating to the physiological signal received from the sensor 14. For example, the patient monitor 12 may include a processor configured to calculate the patient's arterial blood oxygen saturation, tissue oxygen saturation, pulse rate, respiration rate, blood pressure, blood pressure characteristic measure, autoregulation status, brain activity, and/or any other suitable physiological characteristics. Additionally, the patient monitor 12 may include a monitor display 30 configured to display information regarding the physiological parameters, information about the system (e.g., instructions for disinfecting and/or charging the sensor 14), and/or alarm indications. The patient monitor 12 may include various input components 32, such as knobs, switches, keys and keypads, buttons, etc., to provide for operation and configuration of the patient monitor 12. The patient monitor 12 may also display information related to alarms, monitor settings, and/or signal quality via one or more indicator lights and/or one or more speakers or audible indicators. The patient monitor 12 may also include an upgrade slot 28, in which additional modules can be inserted so that the patient monitor 12 can measure and display additional physiological parameters.

Because the sensor 14 may be configured to operate in a wireless mode and, in certain embodiments, may not receive power from the patient monitor 12 while operating in the wireless mode, the sensor 14 may include a battery to provide power to the components of the sensor 14 (e.g., the emitter 16 and the detector 18). In certain embodiments, the battery may be a rechargeable battery such as, for example, a lithium ion, lithium polymer, nickel-metal hydride, or nickel-cadmium battery. However, any suitable power source may be utilized, such as, one or more capacitors and/or an energy harvesting power supply (e.g., a motion generated energy harvesting device, thermoelectric generated energy harvesting device, or similar devices).

As noted above, in an embodiment, the patient monitor 12 is a pulse oximetry monitor and the sensor 14 is a pulse oximetry sensor. The sensor 14 may be placed at a site on a patient with pulsatile arterial flow, typically a fingertip, toe, forehead or earlobe, or in the case of a neonate, across a foot. Additional suitable sensor locations include, without limitation, the neck to monitor carotid artery pulsatile flow, the wrist to monitor radial artery pulsatile flow, the inside of a patient's thigh to monitor femoral artery pulsatile flow, the ankle to monitor tibial artery pulsatile flow, and around or in front of the ear. The patient monitoring system 10 may include sensors 14 at multiple locations. The emitter 16 emits light which passes through the blood perfused tissue, and the detector 18 photoelectrically senses the amount of light reflected or transmitted by the tissue. The patient monitoring system 10 measures the intensity of light that is received at the detector 18 as a function of time.

A signal representing light intensity versus time or a mathematical manipulation of this signal (e.g., a scaled version thereof, a log taken thereof, a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The amount of light detected or absorbed may then be used to calculate any of a number of physiological parameters, including oxygen saturation (the saturation of oxygen in pulsatile blood, SpO2), an amount of a blood constituent (e.g., oxyhemoglobin), as well as a physiological rate (e.g., pulse rate or respiration rate) and when each individual pulse or breath occurs. For SpO2, red and infrared (IR) wavelengths may be used because it has been observed that highly oxygenated blood will absorb relatively less Red light and more IR light than blood with a lower oxygen saturation. By comparing the intensities of two wavelengths at different points in the pulse cycle, it is possible to estimate the blood oxygen saturation of hemoglobin in arterial blood, such as from empirical data that may be indexed by values of a ratio, a lookup table, and/or from curve fitting and/or other interpolative techniques.

As we have noted, exemplary embodiments provide a medical sensor bandage that includes plural transparent wings surrounding at least one medical sensor, the plural transparent wings including an adhesive on a first side and including apertures therethrough configured to provide stretchability in at least one direction to the sides and away from the at least one medical sensor.

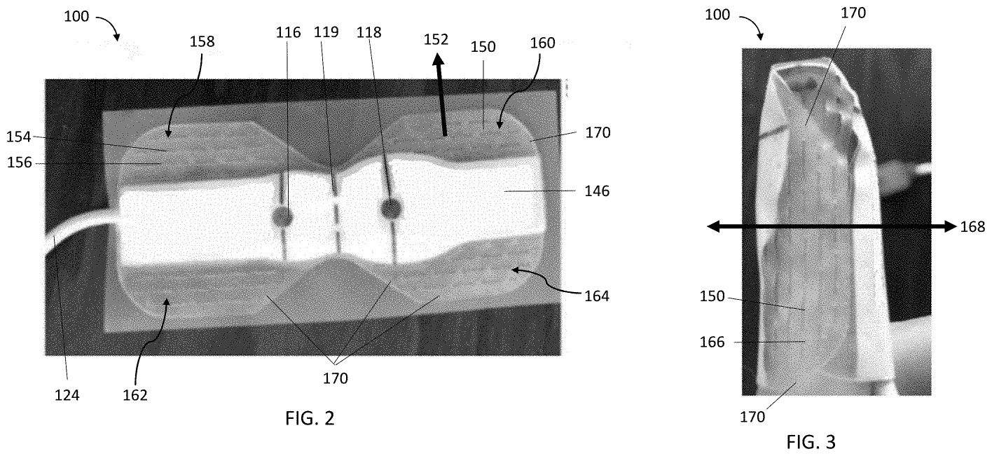

FIG. 2 illustrates an exemplary finger (or other digit) sensor generally at 100. Sensor cable 124 connects to body 146. Markers are shown at 116 and 118, generally showing the position of the emitter(s) and detector(s) (not shown) within the body 146. Fold line 119 generally shows proper positioning for the tip of the finger/digit during patient application.

With further reference to FIG. 2, in exemplary aspects, the apertures include longitudinal slits 150 oriented in a direction perpendicular to the at least one direction of stretchability (illustrated by arrow 152). In exemplary aspects, the slits are knife cut (with or without removal of material), laser cut (with removal of material), or otherwise formed through the bandage material.

In further exemplary aspects, plural rows (e.g., rows 154, 156 in FIG. 2) of longitudinal slits, e.g., 2-4 rows of slits are provided on each wing (note the four wings, shown generally at 158, 160, 162 and 164 in FIG. 2). In further exemplary aspects, the slits in the plural rows are staggered (as shown in FIG. 2) between rows. In further exemplary aspects, the slits are stacked (lined up) between rows. In exemplary embodiments, these slits, or plural rows of slits, are only provided on the plural wings of the bandage. In such embodiments, it can be advantageous to leave out slits on other portions, such as over the sensor body in general, and/or over flex circuits, light blocking layers or optics, to prevent stretch in those areas and/or to prevent a further pathway for ambient light to intrude on light sensitive components or layers.

FIG. 3 illustrates the exemplary sensor bandage 100 of FIG. 2 provided in position over a patient finger 166. Double arrow 168 illustrates the direction(s) of stretch, based upon the orientation of the slits 150.

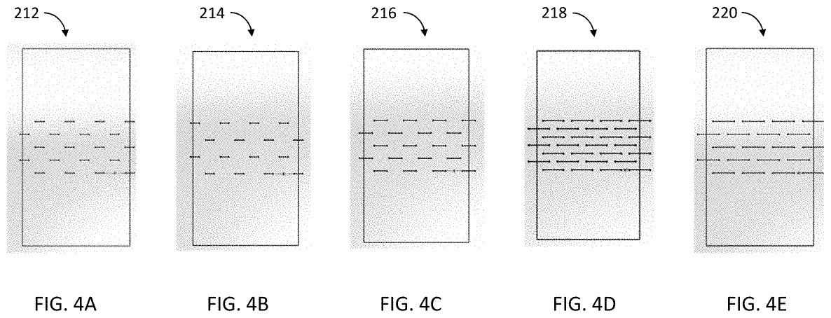

With reference to FIGS. 4A-4E, in exemplary aspects, the slits are provided at nominal lengths between 3 and 5 millimeters (mm), either all with the same length, or with different lengths on the same wings. With reference to the exemplary embodiments shown in FIGS. 4A-4E, exemplary embodiments include, without limitations: slit lengths of 2 mm, 3 mm frequency, staggered (shown generally at 212 in FIG. 4A); slit lengths of 2 mm, 4 mm frequency, staggered (shown generally at 214 in FIG. 4B); slit lengths of 3 mm, 3 mm frequency, staggered (shown generally at 216 in FIG. 4C); slit lengths of 5 mm, 2 mm frequency, staggered (shown generally at 218 in FIG. 4D); and slit lengths of 5 mm, 3 mm frequency, staggered (shown generally at 220 in FIG. 4E), though other slit lengths, frequencies and configurations (staggered vs. stacked), or mixtures thereof are contemplated herein. As we have noted, other embodiments, including variation of plural slits as an arrangement on wings, lengths of slits, numbers of rows, etc., are contemplated herein. For example, a primary factor in increasing strain is staggering of rows, as per the examples above. A secondary factor includes the frequency of rows, with those being more frequent, i.e., closer together, increasing strain. Slit length (longer slits) also increases strain. Accordingly, any particular wing or bandage may be tuned to the application, with those factors in mind.

In exemplary aspects, the slits are provided such that they do not cut through the edge of the bandage wings, thereby providing a constant outer bandage boundary (see the constant edge boundaries 170 in FIGS. 2 and 3) that resists tearing during adhesive liner removal, application to a patient and adjustment/readjustment. In further exemplary embodiments, slit portions are spaced at a minimum distance away from the wing outer boundaries (those boundaries) away from the at least one sensor), e.g., more than about 1 mm, more than about 2 mm, more than about 2.5 mm, more than about 3 mm, more than about 3.5 mm, more than about 4 mm, more than about 4.5 mm, more than about 5 mm, etc.

In exemplary aspects, the sensor bandage includes wings providing stretchability up to 8 mm for a low finger that exerts a low radial pressure (allowing that it is also configured to stretch more for blood pressures higher than, e.g., 40 mmHg). In further exemplary aspects, the sensor bandage includes four wings, with each wing providing stretchability up to 2 mm to accommodate a total radius stretch of 8 mm. In further exemplary aspects, the material strain for the wings is up to about 0.2.

In exemplary aspects, the transparent material is a polyethylene material. In exemplary aspects, the thickness of the polyethylene material is between 0.07 and 0.12 mm thick). In further exemplary aspects, the material is a polyethylene material that is 0.09 mm thick. In further exemplary embodiments, the polyethylene material is acrylic coated. In further exemplary aspects, material is a 0.09 mm acrylic coated polyethylene material. In further exemplary aspects, the geometry of each wing is configured to provide the stretchability described above with an estimation of a 0.6 to 0.75 N axial force applied by a patient finger or other digit. The present application also contemplates other materials and/or other thicknesses of the same or different materials (the properties of which can be accounted for in calculating the relative axial force/strain values for the same application(s) as would be used for the above identified 0.09 mm thick polyethylene material, based on the above and other portions of the specification).

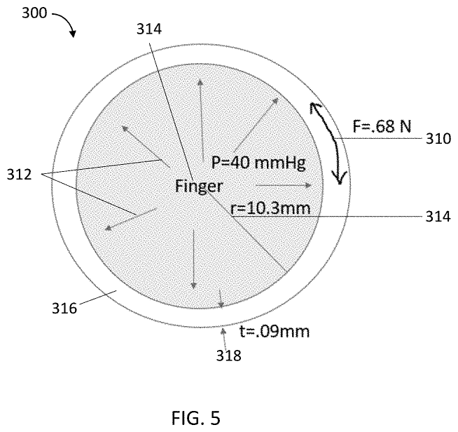

In further exemplary aspects, the geometry of each wing is configured to provide the deflection described above with an estimation of a 0.68 N axial force applied by a patient finger. In further exemplary aspects, the 0.68 N axial force is based upon a diastolic blood pressure of 40 millimeters of mercury (mmHg) applying a hoop force on the bandage, with the bandage able to strain at least 0.2 at the force of 0.68 N. Reference is made to FIG. 5, which illustrates generally at 300 the axial force of 0.68 N (at 310) due to pressure (at 312) from swelling of a finger 314, having a radius of 10.3 mm (at 314) on a bandage material 316 having a thickness of 0.09 mm (at 318). We note that this Figure identifies a strain requirement for a patient, assuming a large finger that swells and induces an axial force of 0.68 N. Additionally, tuning of particular bandage portions (including identifying a particular strain value for patients having specific attributes or an upper strain value for a worst case) may be performed using different assumptions based upon the location of the bandage of the patient, the geometry of that body portion and the axial force that would be applied due to swelling, in a manner similar to the axial force/strain that was calculated for the large finger (having a radius of 10.3 mm) as shown in FIG. 5.

In other exemplary aspects, modeled deflection trends illustrated advantages for slit lengths of 5 mm, staggered, with either two or three rows, though other configurations are contemplated herein.

In further exemplary aspects, the slits provided in the plural wings additionally improve breathability of the bandage, mitigating issues with regard to moist bandages due to diaphoretic patients or other causes of moisture (spills, sweating in general, etc.).

In another aspect, the disclosure provides a patient monitoring pulse oximetry sensor configured to position at least one emitter and at least one sensor over a digit of a patient, which includes the sensor bandage described in exemplary aspects above.

In another aspect, the disclosure provides a patient monitoring system, having a patient monitor coupled to a patient monitoring sensor, which includes the sensor bandage described in exemplary aspects above. The patient monitoring sensor includes a communication interface, through which the patient monitoring sensor can communicate with the patient monitor. The patient monitoring sensor also includes a light-emitting diode (LED) communicatively coupled to the communication interface and a detector, communicatively coupled to the communication interface, capable of detecting light.

Thus, in exemplary embodiments, a one-piece bandage is provided with plural or all layers pre-assembled for manufacture or re-manufacture of the sensor. Exemplary embodiments also facilitate ease of manufacture or re-manufacture and produce reliable and repeatable alignment and contact of the layers, for example by eliminating need to manually align and laminate layers together.

One or more specific embodiments of the present techniques will be described below. In an effort to provide a concise description of these embodiments, not all features of an actual implementation are described in the specification. It should be appreciated that in the development of any such actual implementation, numerous implementation-specific decisions must be made, which may vary from one implementation to another.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

What is claimed is:

1. A bandage for a medical sensor, the bandage comprising:
plural transparent wings configured to surrounding at least one medical sensor, the plural transparent wings comprising:
an adhesive on a first side; and
apertures provided through the transparent wings, the apertures configured to provide stretchability in at least one direction to the sides and away from the at least one medical sensor, wherein the apertures are configured as longitudinal slits oriented in a direction perpendicular to the at least one direction of stretchability.

2. A bandage in accordance with claim 1, wherein the slits are knife cut or laser cut through the bandage material.

3. A bandage in accordance with claim 1, wherein plural rows of longitudinal slits are provided on each wing.

4. A bandage in accordance with claim 1, wherein slits in the plural rows are staggered between rows.

5. A bandage in accordance with claim 1, wherein slits are stacked between rows.

6. A bandage in accordance with claim 1, wherein the slits are provided at nominal lengths between 3 and 5 millimeters (mm), either all with the same length, or with different lengths on the same wings.

7. A bandage in accordance with claim 1, wherein slits are provided such that they do not cut through the edge of the bandage wings, thereby providing a constant outer bandage boundary that resists tearing during adhesive liner removal, application to a patient and adjustment/readjustment.

8. A bandage in accordance with claim 1, wherein slit portions are spaced at a minimum distance away from the wing outer boundaries more than about 1 mm.

9. A bandage in accordance with claim 1, wherein the sensor bandage includes wings providing stretchability up to about 8 mm.

10. A bandage in accordance with claim 1, wherein the sensor bandage includes four wings, with each wing providing stretchability up to 2 mm to accommodate a total radius stretch of 8 mm, with the material strain for the wings is up to about 0.2.

11. A medical sensor, comprising:
at least one medical sensor; and
a bandage, comprising:
plural transparent wings configured to surrounding at least one medical sensor, the plural transparent wings comprising:
an adhesive on a first side; and
apertures provided through the transparent wings, the apertures configured to provide stretchability in at least one direction to the sides and away from the at least one medical sensor, wherein the apertures are configured as longitudinal slits oriented in a direction perpendicular to the at least one direction of stretchability.

12. A bandage in accordance with claim 11, wherein the slits are knife cut or laser cut through the bandage material.

13. A bandage in accordance with claim 11, wherein plural rows of longitudinal slits are provided on each wing.

14. A bandage in accordance with claim 1, wherein slits in the plural rows are staggered between rows.

15. A bandage in accordance with claim 11, wherein slits are stacked between rows.

16. A bandage in accordance with claim 11, wherein the slits are provided at nominal lengths between 3 and 5 millimeters (mm), either all with the same length, or with different lengths on the same wings.

17. A bandage in accordance with claim 11, wherein slits are provided such that they do not cut through the edge of the bandage wings, thereby providing a constant outer bandage boundary that resists tearing during adhesive liner removal, application to a patient and adjustment/readjustment.

18. A bandage in accordance with claim 11, wherein slit portions are spaced at a minimum distance away from the wing outer boundaries more than about 1 mm.

19. A bandage in accordance with claim 11, wherein the sensor bandage includes wings providing stretchability up to about 8 mm.

20. A bandage in accordance with claim 11, wherein the sensor bandage includes four wings, with each wing providing stretchability up to 2 mm to accommodate a total radius stretch of 8 mm, with the material strain for the wings is up to about 0.2.
